(19) United States
(12) Patent Application Publication
(10) Pub. No.: US 2004/0259111 A1
(43) Pub. Date: Dec. 23, 2004

(54) Automated system and method for preparing an assay ready biological sample

(75) Inventors: Jon Carl Marlowe, Bothell, WA (US); Elizabeth Anne West, Seattle, WA (US); Hienthuc Phan, Seattle, WA (US); Emily Ruth Schultz, Bellevue, WA (US)

Correspondence Address: JONES DAY, 222 EAST 41ST ST, NEW YORK, NY, 10017, US

(73) Assignee: Rosetta Inpharmatics LLC

(21) Appl. No.: 10/734,063

(22) Filed: Dec. 10, 2003

Related U.S. Application Data
(60) Provisional application No. 60/432,200, filed on Dec. 10, 2002.
(60) Provisional application No. 60/451,219, filed on Feb. 27, 2003.

(51) Int. Cl. C12Q 1/68; G01N 33/53; G06F 19/00; G01N 33/48; G01N 33/50

(52) U.S. Cl. 435/6; 702/20; 435/7.1

(57) ABSTRACT

Once a binding assay design and sample is received from a scientist, an experiment design is automatically prepared for generating a binding-ready biological sample to be used by the binding assay. Materials usage and plate layout is then automatically optimized for generating the binding-ready biological sample. A robot method is chosen for generating the binding-ready biological sample and work instructions generated for preparing the binding-ready biological sample. The work instructions are based on the experiment design and the robot method. The work instructions are then transmitted towards a controller for execution by robot stations. From the robot method it is then determined whether pooling and/or splitting needs to occur. If pooling and/or splitting needs to occur, a worklist containing a set of instructions for pooling and splitting is generated and transmitted towards the controller for execution by the robot stations.

This application claims benefit to U.S. patent application Ser. No. 60/432,200 filed on Dec. 10, 2002, and U.S. patent application Ser. No. 60/451,219 filed on Feb. 27, 2003, both of which are incorporated herein by reference in their entirety.

### BACKGROUND OF THE INVENTION

1. Field of the Invention

The present invention relates generally to an automated system and method for preparing a biological sample for a binding assay, such as a hybridization assay. More particularly, the invention is preferably directed to an automated system and method for converting, amplifying, purifying, dispensing, quantifying, tagging, and/or labeling a sample to form a binding-ready biological sample to be used in a binding assay.

2. Description of Related Art

Presently, binding assays are used for a wide range of applications such as, gene discovery, disease diagnosis, drug discovery (pharrnacogenomics), and toxicological research (toxicogenomics). One of the most common types of binding assays is the hybridization assay. Hybridization assays are typically used to determine the presence of specific DNA or RNA sequences in a biological sample. The chemical process of hybridization is accomplished by providing a medium for matching known and unknown DNA samples to bind based on base-pairing rules; in which adenine (A) binds to thymine (T) (or uracil [U], in the case of RNA), and cytosine (C) binds to guanine (G). Binding occurs under precisely controlled conditions through the formation of hydrogen bonds between the paired bases, forming the well-known double helix structure.

In general, hybridization assays typically follow the following process. A scientist devises an experiment; he/she obtains a tissue sample, such as by removing organs from a mouse that has been exposed to a potential new drug of interest; a constituent of the tissue sample, such as a nucleic acid (like RNA), is obtained from the tissue sample; this constituent sample is then processed into a hybridization-ready biological sample; an array of known immobilized biological samples (probes) are then contacted with the hybridization-ready biological sample (target) to identify which compounds in the array the target binds, or otherwise reacts, with.

Binding assays, such as hybridization assays, typically utilize microarrays to increase the throughput of the assay. Microarrays generally consist of a substrate, such as a slide or chip made of glass, plastic or silicon, upon which is attached an array of biological probes representing discrete binding or reaction sites for target biological samples. Various types of probes are known, including nucleic acids, proteins, ligands, antibodies or other cellular proteins. For example, a typical DNA microarray includes an array or matrix of DNA probes representing discrete binding sites for at least some of the genes or gene products (e.g., cDNAs, mRNAs, cRNAs, polypeptides, and fragments thereof) in an organism's genome. The layout of these probes may form a single array of thousands of probes across the surface of a single chip (high density array), or the array may be broken into a multitude of identical small arrays (low density) on a single substrate (small array or multiple array format). Other experiment techniques that provide a means to generate similar data to that generated using microarrays include microbead-based assays and direct cDNA sequencing assays.

While microarray, microbead, or direct cDNA assays generally increase the throughput of binding assays, little has been done to increase the speed and throughput of preparing the binding-ready biological samples prior to performing the assay. In fact, even using current robotic systems, it can still take as many as fifteen skilled operators a year to produce only fifty thousand samples. In addition, much of the preparation is still undertaken manually. This lowers assay throughput and increases the probability of human error.

Moreover, once the preparation of the biological sample has been completed, a manual check is typically undertaken to determine whether the correct quality and quantity of binding-ready biological samples were prepared. If the correct quality and quantity of binding-ready biological samples were not prepared, an operator must recalculate the amount of binding-ready biological sample that still needs to be prepared and thereafter redo the preparation. This is both time consuming and inefficient.

Still further, once a binding-ready biological sample has been prepared it rapidly degrades. It is also hard to maintain chain of custody tracking through complicated processing steps.

In light of the above, there is a need for an automated method for preparing binding-ready biological samples, while addressing the above drawbacks of current binding-ready biological sample preparation processes.

### SUMMARY OF THE INVENTION

The present invention provides an automated method for preparing binding-ready biological samples.

According to the invention there is provided a system and method for preparing binding-ready biological samples. Once a binding assay design and sample is received from a scientist, an experiment design is automatically prepared for generating a binding-ready biological sample to be used by the binding assay. Materials usage and plate layout is then automatically optimized for generating the binding-ready biological sample. A robot method is chosen for generating the binding-ready biological sample and work instructions generated for preparing the binding-ready biological sample. The work instructions are based on the experiment design and the robot method. The work instructions are then transmitted towards a controller for execution by robot stations. From the robot method it is then determined whether pooling and/or splitting needs to occur. If pooling and/or splitting needs to occur, then a worklist containing a set of instructions for pooling and splitting is generated and transmitted towards the controller for execution by the robot stations.

As the present invention is automated, the system can preferably operate twenty four hours a day with little or no operator supervision. This leads to increased throughput and capacity, and to a robust system. For example, one hundred binding-ready plates holding the binding-ready biological samples (approximately 9000 samples) can be produced in only thirty two hours. The system also allows for dynamic pooling, splitting, and batching. In addition, the system is compatible with existing inventory, tracking, control, and management systems, and can, therefore, easily incorporate new or altered preparation protocols, where a protocol is an operating procedure for performing a scientific experiment, such as a hybridization assay. This allows for a flexible system that can adapt to new protocols and has a high reliability, which leads to increased productivity. Furthermore, fewer operators are required, thereby reducing operation costs, while reducing tedious, repetitive, and error-prone laboratory tasks. In addition, the above described system and method improves process control, experiment reproducibility, and end-to-end data tracking.

In short, the invention preferably provides a high throughput automation system that performs robust and fully automated RNA amplification, purification, reformatting, and fluorescent dye labeling. The processed samples from this system are preferably used in a dual-color, fluor reversed pair hybridization on microarrays yielding ratio-based data. Sample normalizations and population pooling are preferably performed in real-time.

### BRIEF DESCRIPTION OF THE DRAWINGS

For a better understanding of the nature and objects of the invention, reference should be made to the following detailed description, taken in conjunction with the accompanying drawings, in which:

FIG. 1 is a flow chart of an overview of a binding assay performed according to an embodiment of the invention;

FIG. 2 is a schematic plan view of a system for preparing binding-ready biological samples, according to an embodiment of the invention;

FIG. 3A is a block diagram of the controller shown in FIG. 2;

FIG. 3B is a block diagram of the integration server shown in FIG. 2;

FIG. 3C is a block diagram of the experiment design manager shown in FIG. 2;

FIG. 3D is a block diagram of the LIMS shown in FIG. 2;

FIG. 3E is a block diagram of the database shown in FIG. 2; and

FIGS. 4A-4E are flow charts of a method for preparing binding-ready biological samples, according to an embodiment of the invention.

Like reference numerals refer to corresponding parts throughout the several views of the drawings.

### DETAILED DESCRIPTION OF THE INVENTION

The present invention provides a system and method for preparing multiple binding-ready biological samples for a binding assay, such as a hybridization assay. An exemplary description of preparation of such binding-ready biological samples will now be described.

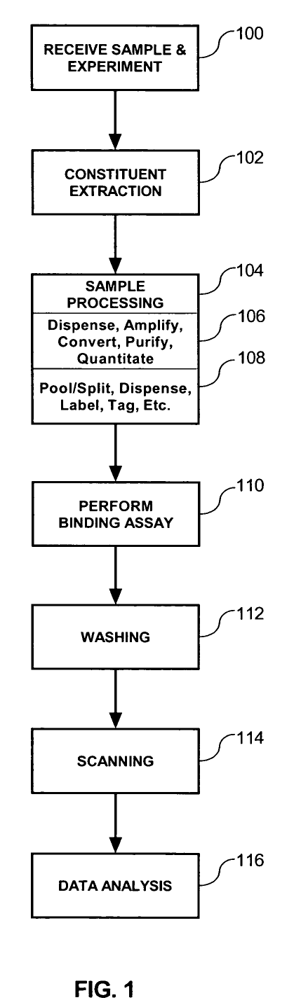

FIG. 1 is a flow chart of an overview of a binding assay performed according to an embodiment of the invention. The binding assay is preferably an array or microarray based experiment. Once a scientist has formulated or devised a binding assay, she sends the binding assay design or plan to the operator responsible for performing the binding assay. The binding assay design or plan is received, at step 100, by the operator together with tissue samples or cell lines on which the assay is to be performed. Typically, a constituent sample, such as a nucleic acid like RNA, is then extracted from the tissue sample, at step 102. The constituent sample is then processed into a binding-ready biological sample, at step 104. Such processing preferably comprises dispensing, amplifying, converting, purifying, and/or quantitating the constituent sample, at step 106. Such processing also preferably comprises pooling, splitting, dispensing, labeling, and/or tagging the constituent sample, at step 108. The binding-ready biological sample is now ready to act as a target in a binding:assay.

The binding-ready biological sample or target is then bound to known probes attached to a substrate, such as a microarray slide or magnetic beads, at step 110. The substrate is subsequently washed, at step 112, to remove any of the target that has not bound to the probes. Next, the substrate is scanned, at step 114, to determine which known probes the target has bound to. Finally, data from the scanning step 114 is analyzed to-ascertain the profile of the target.

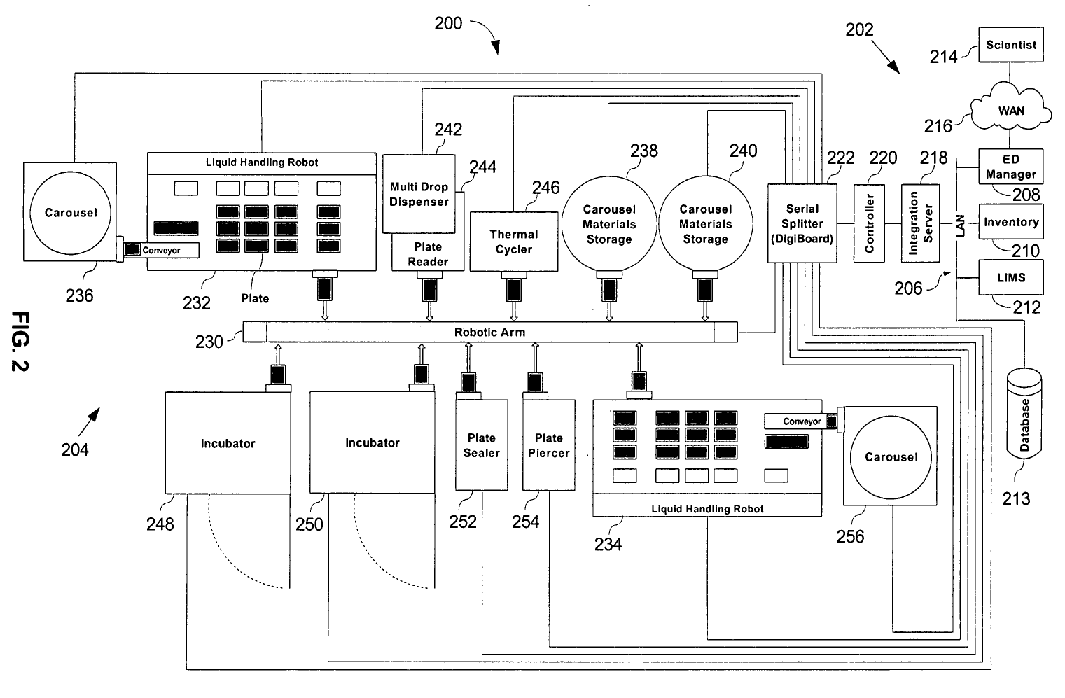

The present invention applies to the sample processing step 104, which is described in detail below in relation to FIGS. 2, 3A-3E, and 4A-4E.

FIG. 2 is a schematic plan view of a system 200 for preparing binding-ready biological samples, according to an embodiment of the invention. The system 200 includes two sub-parts coupled to one another, namely a computer control system 202 and robot stations 204.

The computer control system 202 preferably comprises a Local Area Network (LAN) 206 including an experiment design manager 208, an inventory system 210, a Laboratory Information Management System (LIMS) 212, and a database 213. Although the experiment design manager 208, inventory system 210, LIMS 212, and database 213 are shown as distinct computers, they may be combined together onto one or more computers. However, in a preferred embodiment, the experiment design manager 208, inventory system 210, LIMS 212, and database 213 are distinct servers coupled to the LAN 206.

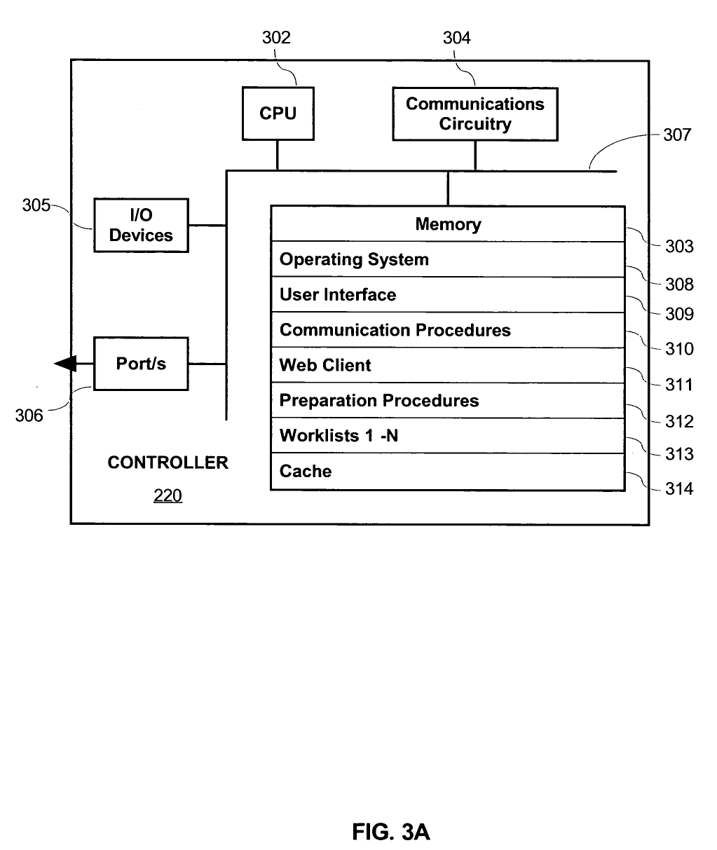

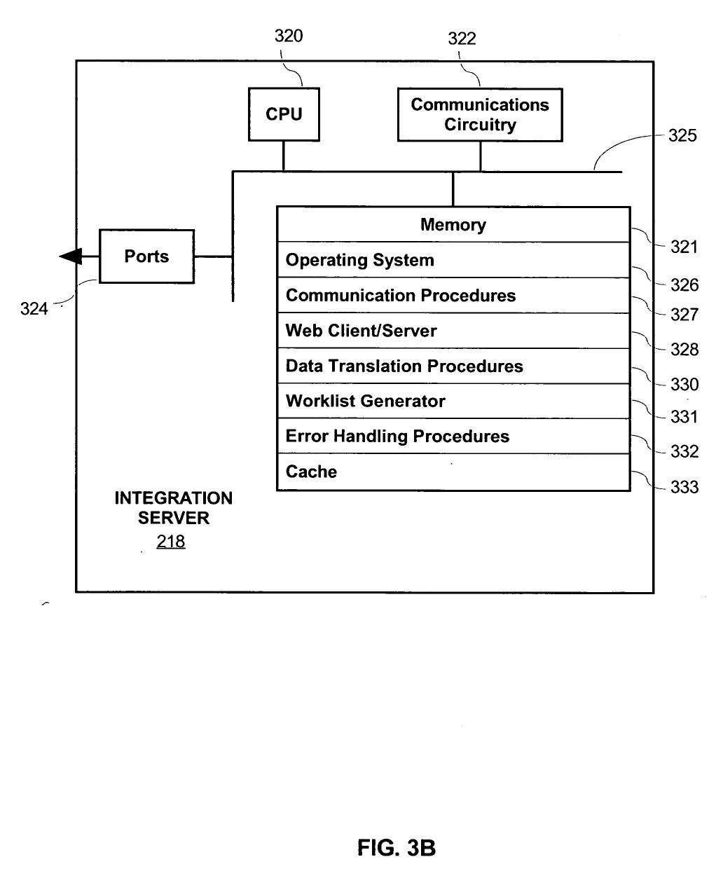

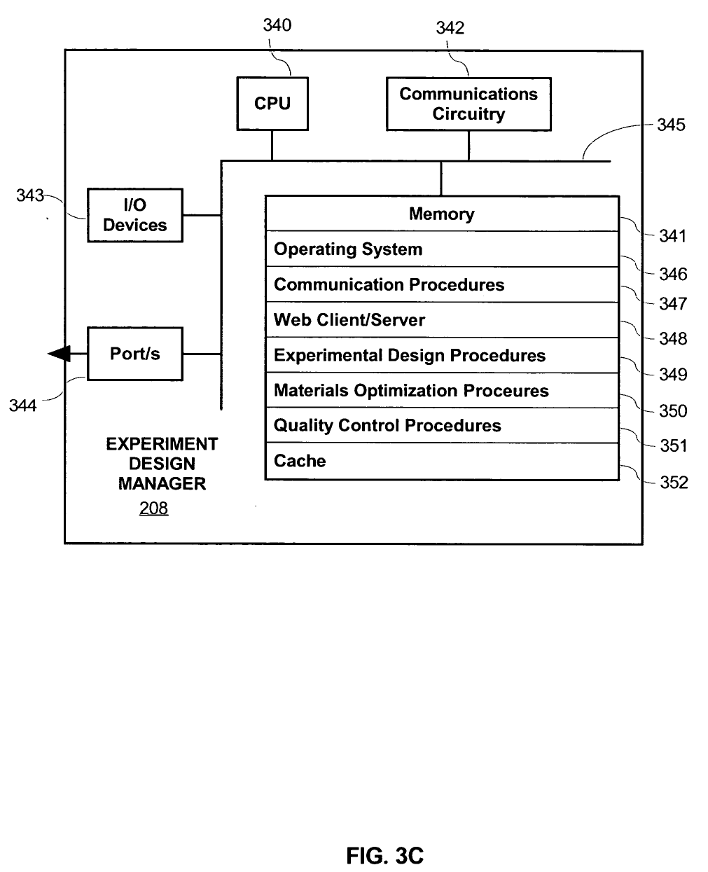

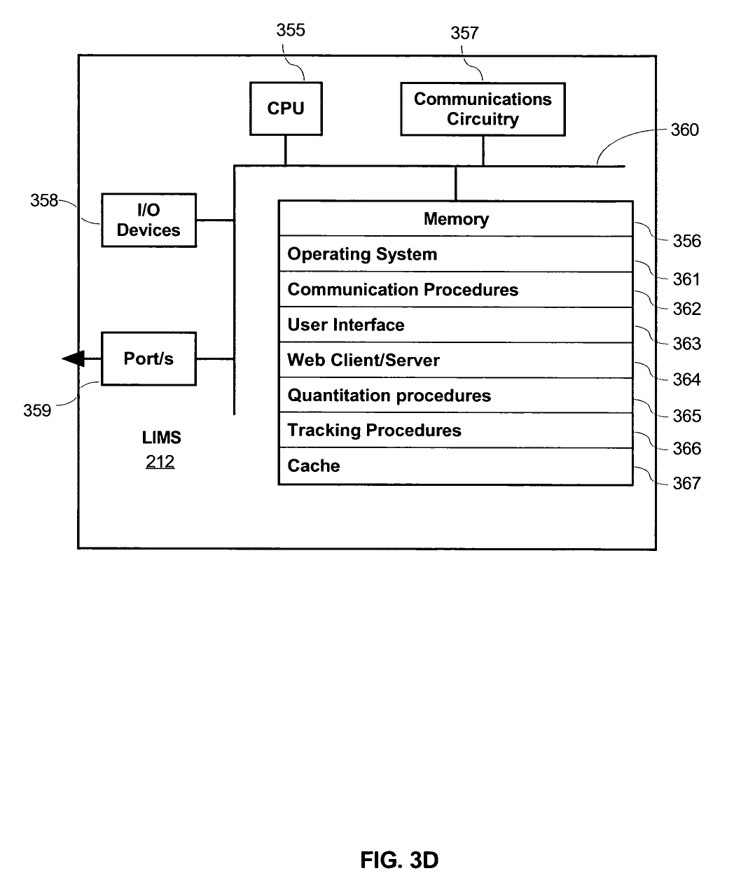

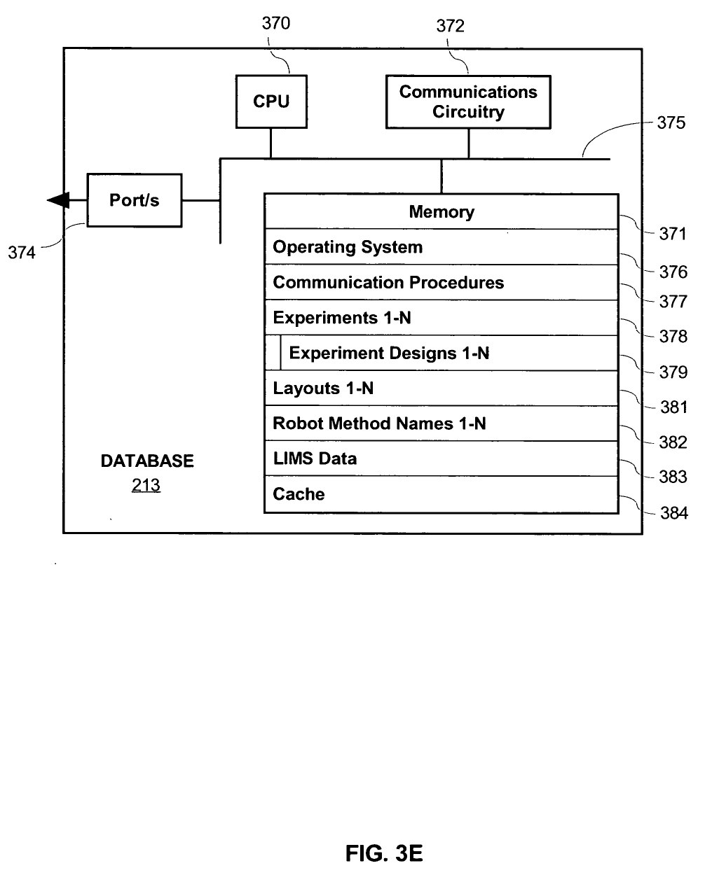

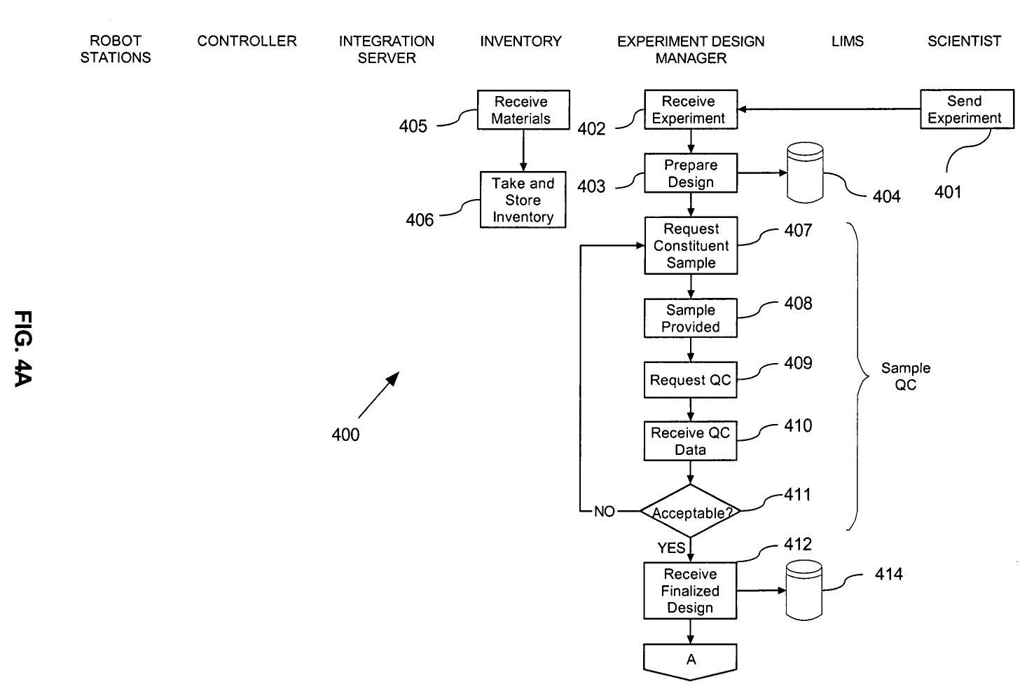

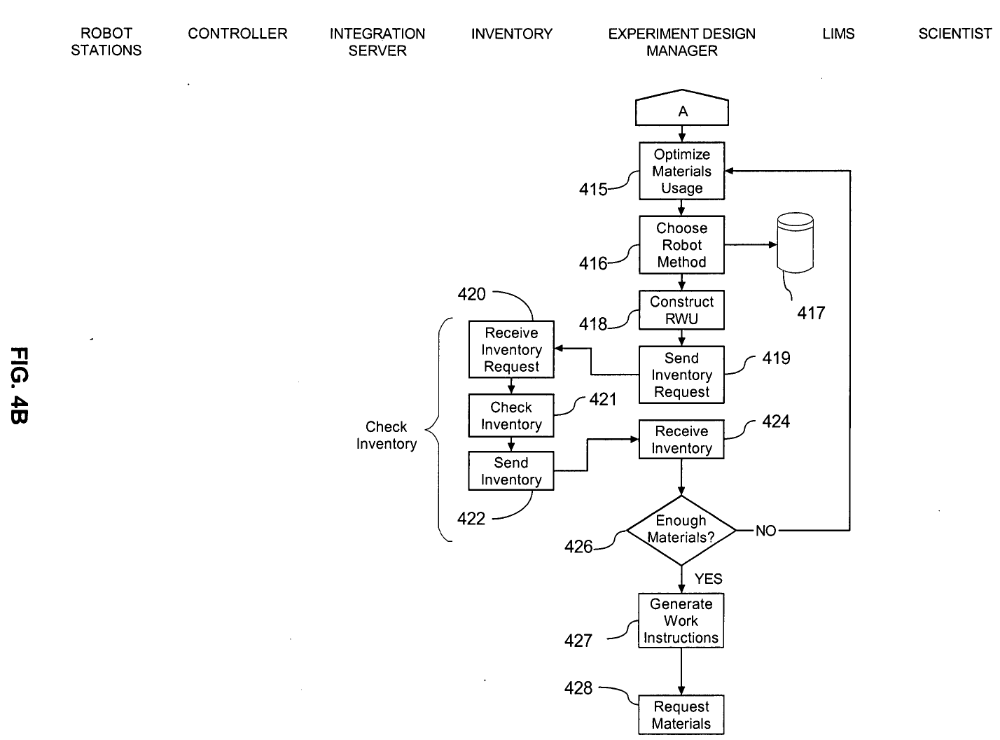

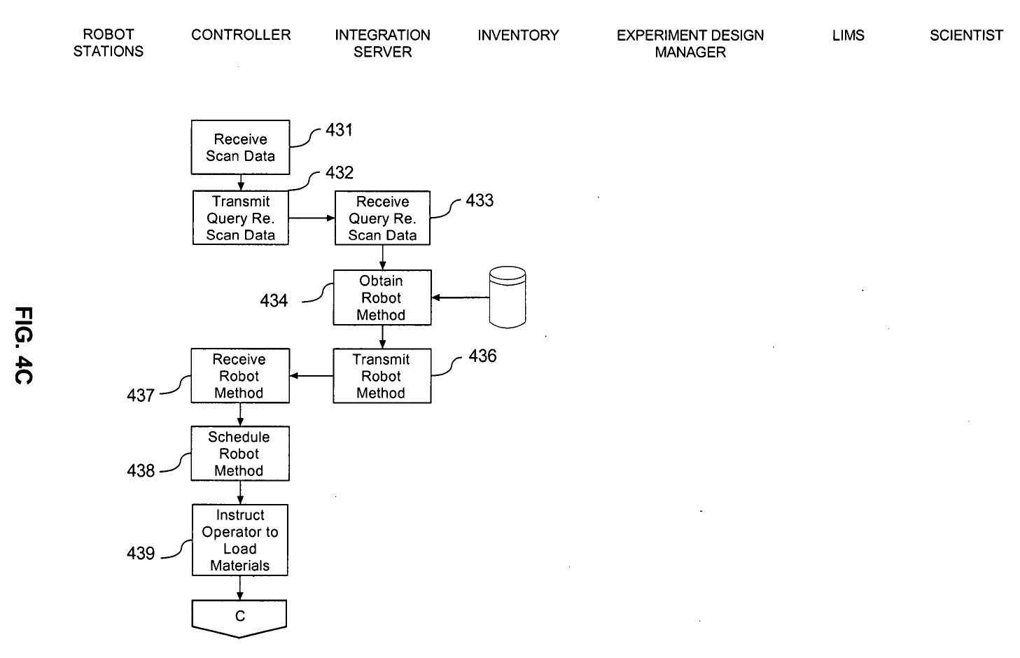

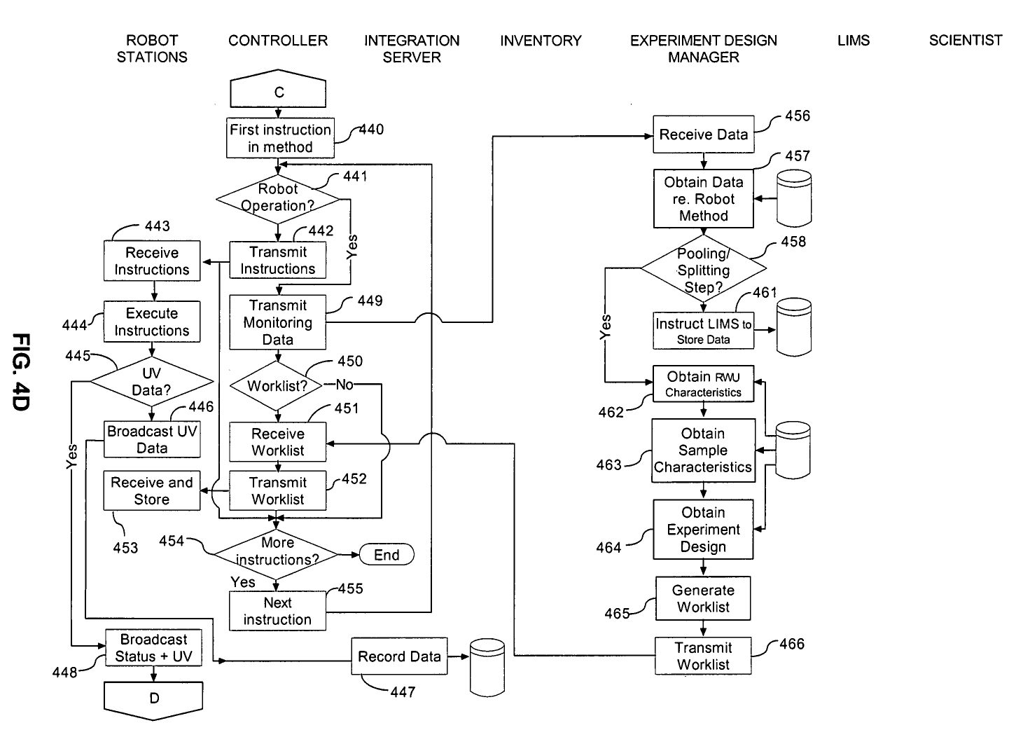

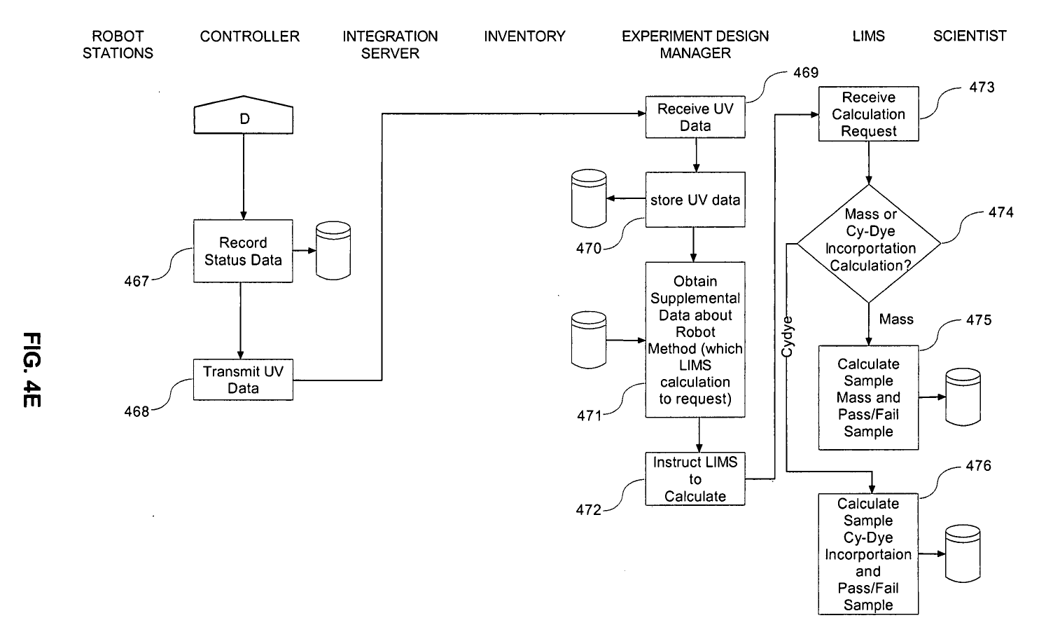

The experiment design manager 208 is used to prepare an experiment design, as described below in relation to FIG. 3C and FIGS. 4A-4E. The experimental design manager primarily consists of software that assists a scientist in designing an experiment, stores the experiment design in a database, schedules the execution of experiments, and groups experiments into Robotic Work Units (RWU). The experiment design manager takes into account the relative priorities of experiments (as indicated by scientists) and availability of parts in inventory when scheduling experiment execution. Furthermore, the experiment design manager groups experiments into RWUs so as to minimize the quantity of materials needed to execute the group of experiments.

A RWU is some nonempty set of experiments. A robot operator loads all the materials needed for executing the experiments comprising a RWU into the robot stations in the same robot loading session. The robot system then processes the materials in the same run. The set of experiments making up the RWU is chosen by the experiment design manager so that as few materials as possible are used during experiment execution. Specifically, the experiment design manager picks experiments for inclusion in a RWU so that the microtiter plates that contain the samples in the experiments are filled as full as possible, thereby minimizing the waste of reagents in unfilled microtiter plate wells.

The inventory system 210 is an inventory management system that tracks all inventory required to produce binding-ready biological samples. Such inventory may include enzymes, plates, disposables, reagents, or the like. A suitable inventory system 210 is made by J. D. EDWARDS. Although not shown, the inventory system hardware preferably includes at least one data processor or central processing unit (CPU); a memory having a database of available inventory; communications circuitry; at least one port that connects to the LAN; and at least one bus that interconnects these components. The inventory system's software stores data indicating which reagents and labware are available for assignment to a RWU, and assigns reagents and labware to RWUs as the experimental design manager creates them.

The LIMS 212 is a system that rapidly collects, delivers, stores and analyzes data during the preparation of the binding-ready biological sample. The LIMS is a relational database system, software Application Program Interface (API), and user interface used to record and retrieve laboratory production data, which is stored in the database 213. The software API provides transactional database interaction. The software API retrieves data, executes programmatic manipulation of the data when required, and writes the data to the database system. The software API also provides functions for data retrieval and data validation. The user interface allows users to access the LIMS 212. In a preferred embodiment, the LIMS 212 is accessed by the integration server 218 using HTTP. The LIMS 212 is described in detail below in relation to FIG. 3D.

The database 213 stores information relating to the preparation of the binding-ready biological sample, as is described in further detail below in relation to FIGS. 3A-3E. Such a database may be housed separately as shown, or may form part of any of the other computer systems described herein. A suitable database is made by ORACLE.

The experiment design manager 208 is also preferably coupled to a scientist client computer 214 via a Wide Area Network (WAN) 216, such as the Internet. This allows a scientist to provide a binding assay plan or design to the experiment design manager 208 from a remote location. Alternatively, the scientist client computer 214 is coupled directly to the LAN 206. In yet another embodiment, the scientist may have the binding assay plan or design entered directly into the experiment design manager 208, such as by being typed into the experiment design manager 208 or by being loaded from a computer disk. However, in a preferred embodiment, the experiment design manager 208 is accessed by a scientist client computer 214 using a web browser, such as INTERNET EXPLORER or NETSCAPE, or through file transfer, such as File Transfer Protocol (FTP).

In addition, an integration server 218 is also preferably coupled between a controller 220 and the LAN, and is preferably accessed from the controller 220 using HTTP. The integration server is preferably a computer system which provides a communication link between the controller 220 and the experiment design manager 208 , LIMS 212, and inventory 210 systems. The integration server 218 compiles incoming/outgoing data and instruction sets and transfers this information to the controller 220, experiment design manager 208, LIMS 212, or inventory 210 systems, as required. The integration server 218 is also preferably used to reformat, reroute and/or interpret commands and other data between the various systems coupled to it. In other words, the integration server essentially translates communications between the various different systems. The integration server 218 is required in a current embodiment, as the various computer systems communicate using different formats and commands. In an alternative embodiment where the systems communicate using the same formats and commands, an integration server 218 may not be necessary. Use of the integration server 218 is described in further detail below in relation to FIG. 3B and FIGS. 4A-4E.

The controller 220 is coupled between the integration server 218 and a serial splitter 222. The controller 220 is preferably a computer that is used to control and schedule preparation of binding-ready biological samples by the robot stations 204. More specifically, the controller 220 preferably provides primary control and communications with the robot stations through a digital serial communications connection between the controller and each specified robot station. The controller 220 parses files stored on the controller into sequences of specific robot station commands, data storage commands, and integration server commands. The controller also schedules the execution of the above sequences, sends commands to the appropriate robot station, stores data, and sends commands to the integration server as it sequentially steps through the schedule it has created. Furthermore, the controller chooses which file/s to parse and execute in response to operator actions and/or commands from the integration server 218.

A serial splitter 222 coupled between the controller 220 and robot stations 204 is preferably used to increase the serial port density of the controller 220. A suitable serial splitter is the DIGIBOARD made by DIGI INTERNATIONAL of Minnetonka, Minn. The serial splitter 222 is preferably coupled to each robot station 204 via separate serial links such as RS232 links. Alternatively, any other suitable link may be used, such as USB 2, wireless, or the like.

The robot stations 204 are custom and/or off-the-shelf programmable laboratory automation devices engineered to perform specific tasks such as liquid handling, labware transfer and positioning, sample and reagent processing, and sample or reagent storage and retrieval, as applied to biological and chemical assays. AS mentioned above, each robot station is preferably independently addressable via digital serial communication from the controller.

A preferred layout of the robot stations 204 includes the following robot stations: a robotic arm 230; liquid handling robots 232 and 234; carousels 236 and 256; two materials storage carousels 238 and 240; a multi-drop dispenser 242; a plate reader 244; a thermal cycler 246; at least two incubators 248 and 250; a plate sealer 252; and a plate piercer 254. It should, however, be appreciated that more or less robot stations arranged in similar or different configurations, may be used.

The various robot stations 204 are preferably centered around the robotic arm 230. In a preferred embodiment, the robotic arm 230 includes an arm controller, has at least 5 degrees of freedom, has at least a 2 kg weight capacity, is robust, and/or is self calibrating. A suitable robotic arm is a three meter ORCA (Optimized Robot for Chemical Analysis) robotic arm made by BECKMAN COULTER. The ORCA robotic arm traverses a rail and can operate on both sides of the rail. The robotic arm 230 is used to transfer materials, such as microtiter plates 237, from robot station to robot station.

The two liquid handling robots 232 and 234 are preferably disposed on opposing sides of the robotic arm 230. One or more of the liquid handling robots 232 and 234 preferably have a 96 channel head and a span-8 head, or two 96 channel. Furthermore, each liquid handling robot 232 has eight pipettors with independent well access. These liquid handling robots also preferably include plate positioning ALPS (Automated Labware Positioning Station), and material storage capabilities. The liquid handling robots 232 and 234 are used for reagent transfers, purifications, pooling, splitting, hybridization loading, or the like. The liquid handling robots 232 and 234 may also perform vacuum-based purification of amplified and florescent-labeled cRNA. A suitable liquid handling robot 232 or 234 is the BIOMEK FX LABORATORY WORKSTATION made by BECKMAN COULTER.

The carousels 236 and 256, as well as the materials storage carousels 238 and 240 are used for storing reagents, chemicals, plates, the constituent sample of the tissue sample, or the like. These carousels preferably each hold one hundred and ninety six full skirted ninety six well plates; ninety deep well blocks; and ninety hybridization carrier fixtures.

The multidrop dispenser 242 is preferably a bulk dispenser. The plate reader 244 is preferably a UV spectrophotometer, such as the SPECTRAMAX PLUS384 made by MOLECULAR DEVICES, which can run both standard spectrophotometer and microplate reader applications on the same instrument. In a preferred embodiment, the plate reader 244 can read 96 or 384 well plates and perform DNA and RNA quantitation.

The thermal cycler 246 preferably has a four plate capacity and is used to perform enzyme inactivations, resuspensions, and Polymerase Chain Reactions (PCR). A suitable thermal cycler 246 is the PELTIER THERMAL CYCLER (PTC-225) DNA ENGINE TETRAD CYCLER made by M J RESEARCH, INC.

Although only two incubators 248 and 250 are shown, the invention preferably includes three incubators, namely 40° C., 42° C., and/or 4° C. incubators capable of holding one hundred and ninety six full skirted ninety six well plates and ninety deep well blocks. These incubators are used for reverse transcription (RT) reactions, in vitro transcription (IVT) reactions, and concentrating samples. In a preferred embodiment, one or both of the incubators 248 and 250 may be a 4° C. storage used for enzyme and reagent storage and preferably holds one hundred and ninety six full skirted ninety six well plates, ninety deep well blocks, and disposable tips. This 4° C. storage incubator is preferably used for enzyme and reagent storage and storage of excess amplified material. It should be appreciated that additional incubators may also be provided. Suitable incubators 248 and 250 include the CYTOMAT line of incubators made by KENDRO LABORATORY PRODUCTS, including the KENDRO CYTOMAT 6000 and 6002. incubators 248 and 250 may be used for a RT and IVT reaction incubations at 40° C. or for concentrating amplified material at 50° C. In addition, in order to achieve rapid concentration of samples, that closely mimics a speed vacuum system, approximately 4 kg of desiccant crystals are preferably placed in the water pan of the incubator to reduce the humidity and increase the rate of evaporation. Using this method, most of the water is removed from the sample wells.

The plate sealer 252 is an automated heat sealer that can seal a wide range of plates, such as full skirted ninety six well plates and deep well blocks, without operator intervention. A suitable plate sealer 252 is the ALPS 300 made by ABGENE. The plate piercer 254 is an automated piercer that can pierce heat seals on full skirted ninety six well plates and deep well blocks. A suitable plate piercer 254 is the ASP 50 (Automated Seal Piercer) also made by ABGENE.

It should be appreciated that because preparation protocols are dynamic and subject to change, the system architecture by necessity is also dynamic. For example, subsystems can be reconfigured or reoptimized, as new technologies become available. Therefore, the system 200 has the flexibility to accommodate changes in experiment preparation protocols.

FIG. 3A is a block diagram of the controller 220 shown in FIG. 2. The controller 220 preferably includes the following components: at least one data processor or central processing unit (CPU) 302; a memory 303; communications circuitry 304 for communicating with the LAN 206 (FIG. 2) and WAN 216 (FIG. 2); input and output devices 305; at least one port 306 that connects to the LAN and the WAN; and at least one bus 307 that interconnects these components.

The memory 303 preferably includes an operating system 308, such as SOLARIS or WINDOWS NT, having instructions for processing, accessing, storing, or searching data, etc. The memory 303 also preferably includes a user interface 309; communications procedures 310 for communicating with the remainder of the system; a web client 311, such as an APACHE server; preparation procedures 312; worklist files 313; and a cache 314 for temporarily storing data.

Suitable preparation procedures 312 are SAMI NT made by BECKMAN COULTER, or other controllers made by CRS BIODISCOVERY. The preparation procedures 312 direct the execution of the sequence of instructions that constitute a “method” or biological sample preparation protocol. The preparation procedures 312 choose how best to interleave the commands to be executed for several batches of samples or plates, possibly being run with different methods. The preparation procedures 312 then step through the instructions in chronological order, communicating with the various robot stations indicated in each command.

A worklist file contains instructions meaningful only to one of the robot stations 204 (FIG. 2). For example, a worklist file may contain pipetting instructions in a format expected by the liquid handling robots. The preparation procedures 312 and worklist files 313 are described in further detail below in relation to FIGS. 4A-4E.

FIG. 3B is a block diagram of the integration server 218 shown in FIG. 2. The integration server 218 preferably includes the following components: at least one data processor or central processing unit (CPU) 320; a memory 321; communications circuitry 322 for communicating with the LAN 206 (FIG. 2) and WAN 216 (FIG. 2); at least one port 324 that connects to the LAN and the WAN; and at least one bus 325 that interconnects these components.

The memory 321 preferably includes an operating system 326, such as SOLARIS or WINDOWS NT, having instructions for processing, accessing, storing, or searching data, etc. The memory 321 also preferably includes: communications procedures 327 for communicating with the remainder of the system; a web client and server 328, such as an APACHE server; data translation procedures 330; a worklist generator 331; error handling procedures 332; and a cache 333 for temporarily storing data. The data translation procedures 330 are used to translate data having different formats between the various computer systems coupled to the integration server. The worklist generator 331 is used to generate worklists, and may be located on the integration server or on any other computer system. The error handling procedures 332 are used to generate and/or handle errors generated by the system.

FIG. 3C is a block diagram of the experiment design manager 208 shown in FIG. 2. The experiment design manager 208 preferably includes the following components: at least one data processor or central processing unit (CPU) 340; a memory 341; communications circuitry 342 for communicating with the LAN 206 (FIG. 2) and WAN 216 (FIG. 2); at least one port 344 that connects to the LAN and/or the WAN; input and output devices 343, such as a keyboard and monitor; and at least one bus 345 that interconnects these components.

The memory 341 preferably includes an operating system 346, such as SOLARIS or WINDOWS NT, having instructions for processing, accessing, storing, or searching data, etc. The memory 341 also preferably includes: communications procedures 347 for communicating with the remainder of the system; a web client and server 348, such as an APACHE server; experiment design procedures 349; materials optimization procedures 350; quality control procedures 351; and a cache 352 for temporarily storing data.

The experiment design procedures 349, optimization procedures 350, and quality control procedures 351 are described below in relation to FIGS. 4A-4E.

FIG. 3D is a block diagram of the LIMS 212 shown in FIG. 2. The LIMS 212 preferably includes the following components: at least one data processor or central processing unit (CPU) 355; a memory 356; communications circuitry 357 for communicating with the LAN 206 (FIG. 2) and WAN 216 (FIG. 2); at least one port 359 that connects to the LAN and the WAN; input and output devices 358, such as a keyboard and monitor; and at least one bus 360 that interconnects these components.

The memory 356 preferably includes an operating system 361, such as SOLARIS or WINDOWS NT, having instructions for processing, accessing, storing, or searching data, etc. The memory 356 also preferably includes: communications procedures 362 for communicating with the remainder of the system; a user interface 363; a web client and server 364, such as an APACHE server; quantitation procedures 365; tracking procedures 366; and a cache 367 for temporarily storing data. Although not shown, the memory 356 may also include an ORACLE database, MACROMEDIA's COLDFUSION, and J2EE. Suitable quantitation procedures 365 and tracking procedures 366 may be found in laboratory software, such as SAPPHIRE made by LABVANTAGE. The quantitation procedures 365 and tracking procedures 366 are described in further detail below in relation to FIGS. 4A-4E.

FIG. 3E is a block diagram of the database 213 shown in FIG. 2. The database 213 is shown as a distinct computer system coupled to the LAN. However, it should be appreciated that the database 213 may be incorporated into any of the computer systems 208, 210, 212, 218, or 220. The database 213 preferably includes the following components: at least one data processor or central processing unit (CPU) 370; a memory 371; communications circuitry 372 for communicating with the LAN 206 (FIG. 2) and WAN 216 (FIG. 2); at least one port 374 that connects to the LAN and the WAN; and at least one bus 375 that interconnects these components.

The memory 371 preferably includes an operating system 376, such as SOLARIS or WINDOWS NT, having instructions for processing, accessing, storing, or searching data, etc. The memory 371 also preferably includes: communications procedures 377 for communicating with the remainder of the system; experiments 1-N 378, including experiment designs 379; layouts 1-N 381; robot method names 1-N 382; LIMS data 383; and a cache 383 for temporarily storing data.

Use of the experiment designs 379, layouts 381, and robot method names 382 are described below in relation to FIGS. 4A-4E. LIMS data 383 tracks all elements of experiment execution, sample transfer, container content, sample genealogy, and sample PASS/FAIL evaluations, as described below.

FIGS. 4A-4E are flow charts of a method 400 for preparing binding-ready biological samples. Initially, as described in relation to FIG. 1, a scientist conceives of and designs a binding assay, such as a hybridization assay or the like. Binding assays include, but are not limited to assays of protein-protein, protein-ligand, protein-DNA, protein-RNA, DNA-RNA, DNA-DNA, RNA-RNA, protein-antibody, or protein-small molecule interactions. In a preferred application, two complementary strands of DNA, or a strand of DNA with a strand of RNA, interact (hybridize) to form a double-stranded nucleic acid molecule. The scientist then sends the binding assay plan or design to the experiment design manager 208 (FIG. 2), at step 401. The binding assay plan or design preferably includes the number of samples, number of arrays, binding assay protocol options, etc.

In a preferred embodiment, the scientist sends the binding assay plan or design to the experiment design manager 208 (FIG. 2) via the scientist client computer 214 (FIG. 2) over the WAN 216 (FIG. 2). For example, the scientist may securely upload the binding assay plan or design via encrypted email, a virtual private network (VPN), or the like. Altematively, the scientist may enter the binding assay plan or design directly into the experiment design manager 208 (FIG. 2) via a keyboard, a computer disk, or the like.

The scientist also preferably supplies a tissue sample or a constituent of the tissue sample to the system 200 (FIG. 2). For example, this tissue sample or constituent may be mailed to an operator of the system. If a tissue sample is received, then a constituent of the physical tissue sample is extracted. For example RNA is extracted from the tissue sample using well know methods and supplied to the system operator. In a preferred embodiment, the constituent is a nucleic acid, such as total RNA that has been isolated from tissue lysates. Data representing the constituent sample and any other materials required for producing the binding-ready biological sample are then entered into the system, where they are received by the inventory system 210 (FIG. 2), at step 405.

The inventory system then takes inventory and stores a list of available inventory in an inventory system database, at step 406.

Once sent by the scientist, at step 401, the binding assay plan or design is received by the experiment design manager's communication procedures 347 (FIG. 3C), at step 402. Based on the received binding assay plan or design, the experiment design procedures 349 (FIG. 3C) then prepare an experiment design for generating the binding-ready biological sample, at step 403. For example, the experiment design procedures 349 (FIG. 3C) determine the processes required to prepare the binding-ready biological sample and the raw materials required to perform these processes. This experiment design is then stored in the database 213 (FIG. 2), at step 404, as an experiment design 379 (FIG. 3E).

The experiment design procedures 349 (FIG. 3C) then request the constituent sample from the operator, at step 407. The constituent sample is provided and the experiment design manager notified, at step 408. Subsequently, the quality control procedures 351 (FIG. 3C) request quality control data for the constituent sample, at step 409. This quality control data is supplied by the operator and received by the experiment design manager at step 410.

The experiment design procedures 349 (FIG. 3C) and the operator then determine from the quality control data whether the constituent sample is acceptable, at step 411. If the constituent sample is not acceptable (411-No), then the experiment design procedures 349 (FIG. 3C) again request a new constituent sample, at step 407, and the process is repeated until an acceptable constituent sample is provided. Alternatively, the operator may elect to continue to step 412 by omitting some unacceptable samples from the experiment design, rather than waiting until the constituent sample is acceptable.

Once an acceptable constituent sample has been provided (411-Yes), the operator is given the opportunity to alter the experiment design parameters. The experiment design procedures 349 (FIG. 3C) then receive a finalized design, at step 412, which is then stored in the database 213 (FIG. 2), at step 414.

Subsequently, the materials optimization procedures 350 (FIG. 3C) optimize materials usage and plate layout, at step 415. The materials may include chemicals, enzymes, reagents, constituent samples, binding-ready biological samples, etc. The materials optimization and layout are then stored in the database 213 (FIG. 2) as a layout 381 (FIG. 3E), at step 417.

The materials optimization procedures 350 (FIG. 3C) also choose a preferred robot method for generating the binding-ready biological sample, at step 416. A robot method is a file resident on the controller and contains an explicit sequence of robot instructions for preparing a set of binding-ready samples according to a predetermined protocol. When executing a scheduled method, the controller steps through the instructions one at a time, transmitting each instruction to the robot station that should carry it out. In one embodiment a default robot method may be chosen, i.e., the experiment design procedures automatically choose a default robot method for preparing the binding-ready biological sample. The name of the robot method is stored in the database, at step 417, as one of the robot method names 1-N 382 (FIG. 3E). The experiment design procedures 349 (FIG. 3C) then construct a Robotic Work Unit (RWU), at step 418, for the particular experiment design, layout, and robot method. A RWU is a set of experiments, one layout, and one robot method name chosen by the experiment design manager's materials optimization procedures, such that the layout of the experiments' samples optimizes materials use. The experiments so chosen must necessarily have the same protocol, as they will be executed by the chosen robot method.

The experiment design procedures 349 (FIG. 3C), using the communication procedures 347 (FIG. 3C), then send an inventory request to the inventory system 210 (FIG. 2), at step 419, to determine whether there is enough inventory to prepare the binding-ready biological sample set out in the experiment design. The inventory system 210 (FIG. 2) receives the inventory request, at step 420, and checks its inventory system database, at step 421, to determine whether the requested materials are available in inventory. The inventory system 210 then sends inventory data back to the experiment design manager 208 (FIG. 2), at step 422, indicating whether the required materials are available in inventory. Altematively, the inventory system 210 (FIG. 2) can check for all available inventory, at step 421, and send back a list of all available materials to the experiment design manager 208 (FIG. 2), at step 422.

The experiment design manager's communication procedures 347 (FIG. 3C) receive the list of inventory, at step 424. The materials optimization procedures 350 (FIG. 3C) then determine, at step 426, whether there are sufficient materials available to prepare the binding-ready biological sample, as required by the experiment design. If there are not enough materials (426-No), then the materials optimization procedures 350 (FIG. 3C) attempt to reoptimize materials usage, at step 415, and the process is repeated until such time as all the required materials are available in inventory, or until the preparation is canceled, which may occur at any time (not shown). The materials optimization procedures 350 (FIG. 3C) may also notify the operator that there are not enough materials to prepare binding-ready biological samples in accordance with the binding assay plan or design. Such a notification preferably includes a list of the required materials that are not currently available in inventory.

A custom user interface allows an operator to initiate the processing of a batch of binding-ready biological samples. The custom interface ensures the following: that batches are initiated only by qualified operators who are allowed to do so; that reagent plates placed in the system contain valid lots of reagent; that sample plates placed in the system actually belong to the indicated batch; that the plates are placed in the correct locations in the carousels; and that batches are only initiated when there is capacity available in the system. The custom user interface accomplishes this by correlating information obtained from the controller with that obtained from the database.

If there are enough materials (426-Yes), then the materials optimization procedures 350 (FIG. 3C) generate work instructions for producing binding-ready biological samples in accordance with the experiment design, robot method, RWU, and available materials, at step 427. The work instructions are directions to the operator of the system. In a preferred embodiment, the work instructions are instructions printed on a piece of paper for use by the operator in manually carrying out ancillary procedures (not shown or described). In accordance with the work instructions, the communication procedures 347 (FIG. 3C) then request the operator to supply the materials to the robot stations 204 (FIG. 2), at step 428.

The operator of the system then individually scans at least one bar-code for the supplied material. The controller's communication procedures 310 (FIG. 3A) receive the scan data, at step 431, and transmit a query regarding the scan data to the integration server 218 (FIG. 2), at step 432. The integration server receives the query regarding the scan data, at step 433, and obtains the robot method 380 (FIG. 3E), at step 434.

The robot method name is then transmitted, at step 436, by the integration server's communication procedures 327 (FIG. 3B) to the controller 220 (FIG. 2). The controller's communication procedures 310 (FIG. 3A) receive the robot method name, at step 437, and the preparation procedures 312 (FIG. 3A) schedule the robot method, i.e., the various processes required to prepare the binding-ready biological samples on the various robot stations 204 (FIG. 4), at step 438. In other words, an automated list or schedule of step-by-step instructions for preparing binding-ready biological samples, as required by the experiment design, is prepared. For example, the controller may schedule the robotic arm 230 (FIG. 2) to grab a microtiter plate that contains the constituent sample, and thereafter transfer the microtiter plate to the liquid handling robot 232 (FIG. 2). The;controller may pause any current projects before recalculating the schedule.

The preparation procedures 312 (FIG. 3A) then instruct the operator to load the various materials required by the experiment design into the specific robot stations 204 (FIG. 2), at step 439. The operator then loads the required materials into the specific robot stations 204 (FIG. 2). These materials may include microtiter plates, disposable tips, enzymes, reagents, the constituent sample of the tissue sample, other chemicals, etc. Alternatively, all the required materials to produce the binding-ready biological sample may already be loaded into the various carousels or incubators 236, 238, 240, 248, 250, and/or 256 (FIG. 2).

The preparation procedures 312 (FIG. 3A) then start with the first instruction in the method, at step 440. The preparation procedures then determine whether the next instruction to be sent to the robot stations is a robot station operation at step 441. If the instruction is a robot station operation (441-Yes), then the instruction is transmitted, at step 442, to the robot station that is to perform the robot operation. In a preferred embodiment, the controller transmits separate instructions to each robot station via the serial splitter 222 (FIG. 2). This instruction is received by the robot station, at step 443, which then executes the instruction at step 444. Such execution preferably includes converting the constituent sample; amplifying the constituent sample; purifying the amplified constituent sample; labeling the constituent sample; pooling and/or splitting the constituent sample; dispensing the constituent sample in microtiter plates; transferring reagents, enzymes, or other liquids; and/or the like. In a preferred embodiment, the robot stations may execute multiple experiment designs simultaneously.

Throughout the process of preparing binding-ready biological samples, aliquots of the sample are analyzed. In a preferred embodiment, the plate reader 244 (FIG. 2) is used to analyze an aliquot of each sample. For example, the plate reader may obtain UV spectrophotometer data used to calculate a sample's mass and quality. Therefore, if the instruction execution does not generate any UV spectrophotometer data (445-No), status data, such as a plate position, is stored in memory 330. UV data is sent to the integration server at step 446. Such UV data is then received and recorded in the database by the integration server at step 447.

If, however, the instruction execution generated UV spectrophotometer data (445-Yes) then UV data as well as status data is broadcast at step 448. This UV and status data is intercepted by the controller, which records the status data in the database for future use at step 467. The UV data is then transmitted towards the experiment design manager at step 468.

The experiment design manager receives the UV data, at step 469, and stores the UV data in the database at step 470. The experimental design procedures 349 (FIG. 3C) in the experiment design manager 208 (FIG. 2) then obtain supplemental data about the robot method from the database at step 471. This obtained robot method notifies the experimental design procedures which LIMS calculation to request, and then the experimental design manager instructs the LIMS to perform an appropriate calculation, at step 472, as determined by the robot method. The LIMS receives the calculation request at step 473. The quantitation procedures 365 (FIG. 3D) in the LIMS 212 (FIG. 2) then determines whether the calculation request is for a mass calculation or for a fluorescent dye incorporation calculation. A mass calculation determines the mass of the sample being prepared, while the fluorescent dye calculation determines how much fluorescent dye to add to the sample preparation at step 474. A suitable fluorescence dye is CYDYE™ which is brand name for a range of fluorescence dyes, so called as they evolved from a dye class called the cyanines. It should, however, be appreciated that these calculation requests are merely exemplary and in use any other suitable calculation may be performed.

If the calculation request is for mass calculation (474-Mass), then the quantitation procedures calculate the sample mass from the UV data and set a PASS/FAIL evaluation status value, at step 475, i.e., whether enough sample was prepared in accordance with the experiment design. The sample mass and PASS/FAIL is stored in the database and an operator may be notified if not enough sample was prepared. Anything with an evaluation of FAIL may not be used in any further processing.

Similarly, if the calculation request is for a fluorescent dye incorporation calculation (474-CYDYE), then the quantitation procedures calculate the CYDYE incorporation and set a PASS/FAIL evaluation status value at step 476. The CYDYE incorporation and PASS/FAIL evaluation status value is stored in the database and an operator may be notified if a failure occurred.

If the instruction is a not a robot station operation (441-No) (FIG. 4D), then the controller transmits data derived from monitoring the status of the robot stations, such as plate locations, along with data about the progress of the method, to the experiment design manager at step 449. This data is received by the experiment design manager, at step 456, which then obtains supplemental data about the robot method from the database, at step 457, and subsequently determines if the current robot method step is a pooling and/or splitting step, at step 458. Pooling is a process by which several samples or controls (or portions thereof) are mixed to form a common, homogeneous control, called the “pool.” Aliquots of the pool are split-out (splitting) so that each experiment or sample has a corresponding control, thus forming a sample-control pair.

In a preferred embodiment, the controller actually sends data to the integration server, which then sends it to the LIMS, database, and/or experiment design manager. The LIMS and experiment design manager may then send the data to the database.

If the current method step is not a pooling/splitting step (458-No), then the experiment design manager instructs the LIMS to store the data at step 461. LIMS tracks all sample locations in containers and all sample genealogies.

If the current method step is a pooling/splitting step (458-Yes), then the experiment design procedures 349 (FIG. 3C) of the experiment design manager 208 (FIG. 2) obtain RWU characteristics from the database, at step 462, such as a list of all the separate experiments being processed on the set of plates currently being manipulated by the robot stations. The experiment design procedures also obtain sample characteristics from the database, at step 463, such as mass or CYDYE incorporation data. Also, the experiment design procedures obtain the experiment design from the database, at step 464. The experiment design procedures (or the worklist generator 331 (FIG. 3B) on the integration server) then generate a worklist (worklist file 313—FIG. 3A) for pooling and/or splitting at step 465. This worklist is transmitted to the controller, at step 466, which receives the worklist at step 451. The worklist is a set of instructions used for pooling and/or splitting and other single robot operations. The worklist is then transmitted to the robot stations, at step 452. The robot stations receive the worklist and store the worklist for execution in a future instruction, at step 453.

Once the monitoring data has been transmitted to the experiment design manager, at step 449, the preparation procedures 312 (FIG. 3A) of the controller 220 (FIG. 2) determine whether the instructions indicate that a worklist is to be generated, i.e., pooling and/or splitting, at step 450. If a worklist is to be generated (450-Yes), then the controller waits to receive a worklist, and thereafter receives a worklist, at step 451, as described above.

Once a worklist has been transmitted to the robot stations, at step 452, or if it is determined that no worklist is to be generated (450-No), then the preparation procedures determine whether there are any more instructions at step 454. If there are no more instructions (454-No), then the process ends. However, if there are more instructions (454-Yes), then the current instruction is the next instruction in the method, at step 455, and steps 441 onward are repeated.

The following example is used to better illustrate the present invention.

### EXAMPLE

The microarray sample preparation process automated by the system was the RT-IVT and CYDYE™ labeling protocol. Further details of this protocol can be found in U.S. Pat. No. 6,132,997, which is incorporated herein by reference. The automated system can process up to 9600 samples at one time in batches of ten 96-well plates. A new batch can be loaded onto the system and started every hour. In a normal eight hour day 9 batches can be processed on the full automation system at one time. Each batch requires 25 and one half hours to complete. Therefore, up to 8,600 samples can be prepared for hybridization per day.

To perform the RT-IVT amplification protocol, 5 μg of total RNA for each sample enter the system dried down with primers and spike-ins in sealed 96-well PCR plates. Before the samples are dried down and sealed, internal controls and primers are added to each sample. In order to perform the RT reaction the total RNA samples are rehydrated on the BIOMEK FX by the addition of RNase free water. After the primers are annealed by incubation at 65° C. in the M J RESEARCH TETRAD, the BIOMEK FX adds the sample to a plate containing reverse transcriptase. The plate is sealed on the ABGENE ALPS 300 and placed into the 40° C. KENDRO CYTOMAT™ incubator by the ORCA arm. After the two hour RT incubation the RT enzyme is inactivated at 90° C. using a thermal cycling sequence at 90° C. for five minutes in the M J RESEARCH TETRAD. The plate is then pierced on the ABGENE ASP 50 and transported to the dual 96 BIOMEK FX where the samples are transferred into T7 polymerase reagent ready plates. The sample plates are sealed once again on the ABGENE ALPS 300. The 16-hour IVT reaction incubation takes place at 40° C. in a KENDRO CYTOMAT™ incubator. After the IVT incubation is complete, the T7 polymerase enzyme is denatured for 5 minutes at 90° C. in the MJ RESEARCH TETRAD PCR engine. The fully automated system completes the RT-IVT amplification process for each batch of 10 plates in 19 hours.

Purification of the amplified cRNA occurs on the dual 96 channel BIOMEK FX. The purification process involves a MILLIPORE MONTAGE™PCR96 filter plate system. The dual 96 channel BIOMEK FX is equipped with 4 BECKMAN vacuum control units that allow for up to 4 purifications at one time. Purification of 10 plates occurs in less than 60 minutes.

After the cRNA purification process, the optical density at 260 nm and 280 nm is measured for each sample on the MOLECULAR DEVICES SPECTRAMAX³⁸⁴. The calculated sample concentration is combined with the experimental design to achieve representative experimental populations at a desired concentration or total mass. The samples population or pools are later combined with individual samples to achieve a self versus reference two-channel hybridization. The system software, using the calculated concentration, dynamically creates a worklist instruction set in run time for the Hybrid BIOMEK FX. When necessary the hybrid BIOMEK FX creates sample pools of up to 4 ml or approximately 40 individually amplified samples. Once the sample pools are created, the system software generates a second worklist instruction set to create the coupling ready plate sets. A coupling ready plate is one of two plates in a set that will be labeled with separate fluorescent dyes then combined into one plate to achieve a fluor reverse pair. A single coupling ready plate is a plate that contains aliquots of individual samples or pools all at a uniform total mass of 5 μg. Creating a coupling ready plate takes approximately one hour to complete per batch.

Once the coupling ready plate set is created, the un-sealed coupling ready plates are dried at 50° C. in a KENDRO CYTOMAT® incubator for one hour. The samples must be concentrated and re-hydrated at a uniform volume to ensure that all of the samples are at the same working concentration for the labeling reaction. To achieve a low humidity and reduce the time for total evaporation to occur, the incubator water tray is filled with DRY RITE desiccant material. The incubation at 50° C. completely evaporates a sample with a starting volume of 25 μL or less in approximately 90 minutes. After the coupling ready plate set is concentrated the samples are resuspended in 0.1M Bicarbonate Buffer (pH 7.7-8.1) on the dual 96 channel BIOMEK FX.

The fluorescent dyes are added to the coupling ready plate set on the dual 96 channel BIOMEK FX. After the labeling incubation at room temperature, the two channels are combined into one plate and the labeled material is purified using the MILLIPORE MONTAGE™ PCR96 filter plate system. Further details of purifying Nucleic Acid molecules can be found in Co-pending U.S. application Ser. No. 60/513,933, filed on Oct. 24, 2003, which is incorporated herein by reference.

The percent dye incorporation and labeled RNA concentration is measured using the MOLECULAR DEVICES SPECTRAMAX³⁸⁴. Chemical fragmentation occurs by the addition of reagents on the dual 96 channel BIOMEK FX. Once the fragmentation is complete, the hybridization-ready plates are sealed and stored at 4° C. in a KENDRO CYTOMAT® incubator. An operator removes the hybridization-ready plates and performs the addition of the samples to the microarray on a stand alone hybridization loading workstation.

The processing of the samples is performed on the fully automated system using a cascade plate processing approach and Bioware. Bioware are plates of costly reagents in the appropriate working volume that are ready for automation use. They reduce operating costs and streamline the automation processing. The Bioware reagent ready plate contains the working volume or the actual volume of reagent or enzyme that is required for the chemical reaction. A process improvement is to transfer the sample material from the current plate into the Bioware plate. The Bioware plate now contains sample and other reagents previously added to the sample plus the reagent or enzyme that was originally in the plate. Every plate is sealed prior to being placed on the system. After a reagent addition, the 96 well plate is sealed for incubations and thermal cycling. The cascade processing approach was developed to overcome the challenge of automated sealing and piercing of a single plate multiple times as well as streamline the reagent plate use on the full automation system. Further details for storing compositions useful for synthesizing Nucleic Acid Molecules can be found in co-pending U.S. application Ser. No. 60/495,977, filed on Aug. 18, 2003, and incorporated herein by reference.

RT-IVT yield data was generated using 5 μg of total RNA from JURKAT and K562 cell lines as input. The average yield for 16 samples of JURKAT and K562 using the manual workstation protocol is 46.4 μg and 44.6 μg, respectively. The average yield for 16 samples of JURKAT and K562 on the full automation system is 47.9 μg and 47.7 μg, respectively. The CV of the amplification yield for all sample is less than 5% of the average yield.

Fluor reverse pair hybridizations were performed with samples prepared on the manual workstations and the full automation system. Hybridization sensitivity and specificity of samples prepared on the full automation system are equivalent with a P value of 0.01.

In this way, total RNA samples are amplified, purified, quantitated, pooled and split, labeled, and fragmented on the full automation system in an end-to-end process fashion. Custom software and a LIMS database track and drive each automation transaction that occurs on each sample run on the full automation system. Samples processed on the flexible fully automated system have amplification and hybridization data that is comparable to samples processed on the manual workstation platform.

The annual processing capacity of a system such as this is at least 40 fold greater than the capacity of today's microarray laboratories. The increase in sample processing capacity will drive development of 96-well microarray platforms and high throughput automation technologies for down stream processing such as microarray hybridization processing and scanning.

Accordingly, the above described system can operate for longer periods, requires fewer operators, and produces significantly more binding-ready biological samples than the prior art. For example, previously it would take fifteen operators a year to produce fifty thousand binding-ready biological samples. Now, using the above described system, it will take seven operators a year to produce five hundred thousand binding-ready biological samples.

The foregoing descriptions of specific embodiments of the present invention are presented for purposes of illustration and description. They are not intended to be exhaustive or to limit the invention to the precise forms disclosed. Obviously many modifications and variations are possible in view of the above teachings. For example, any of the aforementioned computers or robot stations, may be combined with one another. Also, calculations performed on computer system may be performed on other computer systems, or the calculation spread between computer systems. The embodiments were chosen and described in order to best explain the principles of the invention and its practical applications, to thereby enable others skilled in the art to best utilize the invention and various embodiments with various modifications as are suited to the particular use contemplated. Furthermore, the order of steps in the method are not necessarily intended to occur in the sequence laid out. It is intended that the scope of the invention be defined by the following claims and their equivalents.

What is claimed is:

1. A computer implemented method for preparing a binding-ready biological sample for a binding assay, comprising:
receiving a binding assay design for a binding assay;
preparing an experiment design for generating a binding-ready biological sample to be used in said binding assay;
optimizing materials usage and plate layout for generating said binding-ready biological sample;
choosing a robot method for generating said binding-ready biological sample;
generating work instructions for generating said binding-ready biological sample based on said binding assay design and said robot method; and
transmitting the work instructions towards a controller for execution by robot stations.

2. The method of claim 1, further comprising:
determining from said robot method that pooling and splitting needs to occur;
generating a worklist containing a set of instructions for pooling and splitting; and
transmitting the worklist towards the controller for execution by the robot stations.

3. The method of claim 1, further comprising:
receiving UV spectrophotometer data for an at least partially prepared sample of said binding-ready biological sample;
determining which calculation to perform using said UV spectrophotometer data, from said robot method;
instructing a Laboratory Information Management System (LIMS) to perform said calculation.

4. The method of claim 3, further comprising calculating a mass of said at least partially prepared sample.

5. The method of claim 4, further comprising determining whether said mass of said at least partially prepared sample is sufficient to perform said binding assay.

6. The method of claim 3, further comprising calculating fluorescent dye incorporation for said at least partially prepared sample.

7. The method of claim 6, further comprising determining whether said fluorescent dye incorporation is sufficient to perform said binding assay.

8. The method of claim 1, further comprising executing said work instructions on robot stations to generate said binding-ready biological sample.

9. The method of claim 8, wherein said executing includes processes selected from a group consisting of: converting; amplifying; purifying; dispensing; quantifying; tagging; labeling; transferring reagents, enzymes, or other liquids; pooling; splitting; and any combination of the aforementioned.

10. The method of claim 1, further comprising, before said generating, checking inventory for materials required for said experiment design.

11. The method of claim 10, wherein said checking comprises:
sending a inventory request to an inventory system, where said inventory request contains a list of materials required for said preparation;
receiving inventory data indicating whether said materials are available in inventory; and
ascertaining from said inventory data whether said materials are available in inventory.

12. The method of claim 10, wherein said checking comprises: sending a inventory request to an inventory system;
receiving a list of all materials available in inventory;
ascertaining whether there are enough materials in inventory for said experiment design.

13. The method of claim 1, wherein said binding-ready biological sample is a hybridization-ready biological sample, and said binding assay is a hybridization assay.

14. A computer implemented method for preparing a binding-ready biological sample for a binding assay, comprising:
receiving a binding assay design for a binding assay;
preparing an experiment design for generating a binding-ready biological sample to be used in said binding assay;
choosing a robot method for generating said binding-ready biological sample;
generating work instructions for generating said binding-ready biological sample based on said experiment design and said robot method; and
executing said work instructions on robot stations to generate the binding-ready biological sample.

15. The method of claim 14, further comprising, before said generating, optimizing materials usage and plate layout for generating said binding-ready biological sample.

16. The method of claim 14, further comprising, before said generating, checking inventory for materials required for said experiment design.

17. The method of claim 16, wherein said checking comprises:
sending a inventory request to an inventory system;
receiving a list of all materials available in inventory;
ascertaining whether there are enough materials in inventory for said preparation.

18. The method of claim 16, wherein said checking comprises:
sending a inventory request to an inventory system, where said inventory request contains a list of materials required for said preparation;
receiving inventory data indicating whether said materials are available in inventory; and
ascertaining from said inventory data whether said materials are available in inventory.

19. The method of claim 18, wherein said ascertaining comprises:
concluding that there are not enough materials in inventory for said preparation;
notifying an operator that there are insufficient materials in inventory; and
repeating said ascertaining until there are enough materials in inventory for said preparation.

20. The method of claim 14, wherein said receiving further comprises acquiring a tissue sample.

21. The method of claim 20, further comprising, after said acquiring:
extracting a constituent sample from said tissue sample; and
updating inventory to include said constituent sample.

22. The method of claim 14, wherein said binding-ready biological sample is a hybridization-ready biological sample, and said binding assay is a hybridization assay.

23. A system for preparing a binding-ready biological sample for a binding assay, comprising:
multiple robot stations configured for preparation of a binding-ready biological sample;
a controller for controlling said multiple robot stations; and
a experiment design manager that communicates with said multiple robot stations, said experiment design manager comprising:
a data processor;
communications circuitry for communicating with said multiple robot stations;
input and output devices;
at least one port coupled to said multiple robot stations; and
a memory, comprising:
instructions for receiving a binding assay design for a binding assay;
instructions for preparing an experiment design for generating a binding-ready biological sample to be used in said binding assay;
instructions for optimizing materials usage and plate layout for generating said binding-ready biological sample;
instructions for choosing a robot method for generating said binding-ready biological sample;
instructions for generating work instructions for generating said binding-ready biological sample based on said experiment design and said robot method; and
instructions for transmitting the work instructions towards said controller for execution by said robot stations.

24. The system of claim 23, further comprising additional components selected from a group consisting of: an inventory system, a Laboratory Information Management System (LIMS), a database, an integration server, a serial splitter, a scientist computer, and any combination of the aforementioned components.

25. The method of claim 23 wherein said binding-ready biological sample is a hybridization-ready biological sample, and said binding assay is a hybridization assay.
